(19) United States
(12) Patent Application Publication
(10) Pub. No.: US 2011/0144531 A1
(43) Pub. Date: Jun. 16, 2011

(54) Gastric Band System Injector with Accelerometer

(76) Inventors: Amy L. Marcotte, Mason, OH (US); Daniel F. Dlugos, JR., Middletown, OH (US)

(21) Appl. No.: 12/639,633

(22) Filed: Dec. 16, 2009

(51) Int. Cl. A61B 5/00; A61F 2/00

(52) U.S. Cl. 600/561; 600/37

(57) ABSTRACT

A pressure sensor may be used to determine the pressure of fluid within an implantable gastric restriction device. An accelerometer may be used to measure movement of the patient in which the gastric restriction device is implanted, such that the movement may be accounted for when evaluating data from the pressure sensing system. For instance, movement data from the accelerometer may be used to identify pressure data that should be ignored. A display may annotate a pressure graph to show changes in pressure associated with patient movement; or may otherwise influence the display of pressure data based on patient movement. The pressure sensor and accelerometer may be incorporated into an adapter placed between a syringe and needle that are used to adjust the fluid pressure in the gastric restriction device. Upon examination of pressure data in relation to movement data, the implantable gastric restriction may be adjusted as needed.

### BACKGROUND

A variety of systems and devices have been made and used for treating morbid obesity. Some such systems and devices include adjustable gastric band systems, which are operable to restrict the flow of food from the esophagus into the stomach. Some gastric bands include a fluid-filled elastomeric bladder with fixed endpoints that encircles the stomach just inferior to the gastro-esophageal junction. When fluid is added to the bladder, the band expands against the stomach, creating a food intake restriction or stoma in the stomach. To decrease this restriction, fluid is removed from the bladder. Examples of gastric bands are disclosed in U.S. Pat. No. 7,416,528, entitled “Latching Device for Gastric Band,” issued Aug. 26, 2008, the disclosure of which is incorporated by reference herein.

In some settings, it may be desirable to obtain data indicative of the pressure of fluid in a gastric band. Various examples of methods and devices for obtaining pressure data are disclosed in U.S. Pub. No. 2006/0189888, entitled “Device for Non-Invasive Measurement of Fluid Pressure in an Adjustable Restriction Device,” published Aug. 24, 2006, the disclosure of which is incorporated by reference herein. Such pressure data may be used to determine whether the amount of fluid in the gastric band needs to be adjusted; and/or for other purposes.

While a variety of gastric band systems have been made and used, it is believed that no one prior to the inventor(s) has made or used an invention as described herein.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements and in which:

FIG. 1 depicts a perspective view of an implantable portion of an exemplary gastric band system;

FIG. 2 depicts a perspective view of the gastric band of FIG. 1, showing the band positioned around the gastro-esophageal junction of a patient;

FIG. 3 depicts a cross-sectional view of the gastric band of FIG. 1, showing the band positioned around the gastro-esophageal junction of a patient in a deflated configuration;

FIG. 4 depicts a cross-sectional view of the gastric band of FIG. 1, showing the band positioned around the gastro-esophageal junction of a patient in an inflated configuration to create a food intake restriction;

FIG. 5 depicts a perspective exploded view of an exemplary pressure sensing syringe system usable with the gastric band system of FIG. 1;

FIG. 6 depicts a graphical representation of fluid pressure data plotted as a function of time;

FIG. 7 depicts a perspective exploded view of an exemplary pressure sensing component usable with the gastric band system of FIG. 1;

FIG. 8 depicts a cross-sectional view of the pressure sensing component of FIG. 7;

FIG. 9 depicts a cross-sectional view of an exemplary alternative pressure sensing component usable with the gastric band system of FIG. 1, coupled with a reusable cable;

FIG. 10 depicts a cross-sectional view of the alternative version of the pressure sensing component, showing only the interface portion of the reusable cable;

FIG. 11 depicts a cross-sectional view of an exemplary alternative version of the interface portion of the reusable cable depicted in FIG. 7;

FIG. 12 depicts a graphical representation of pressure data as a function of time with various annotations;

FIG. 13 depicts alternative version of a graphical representation of pressure data as a function of time;

FIG. 14 depicts a graphical representation of pressure data as a function of time with an overlay depicting accelerometer data;

FIG. 15A depicts a block schematic view of an accelerometer and a needle inserted into a patient, with the patient lying in a supine position; and

FIG. 15B depicts a block schematic view of the accelerometer and needle of FIG. 15A, with the patient sitting or standing in an upright position.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

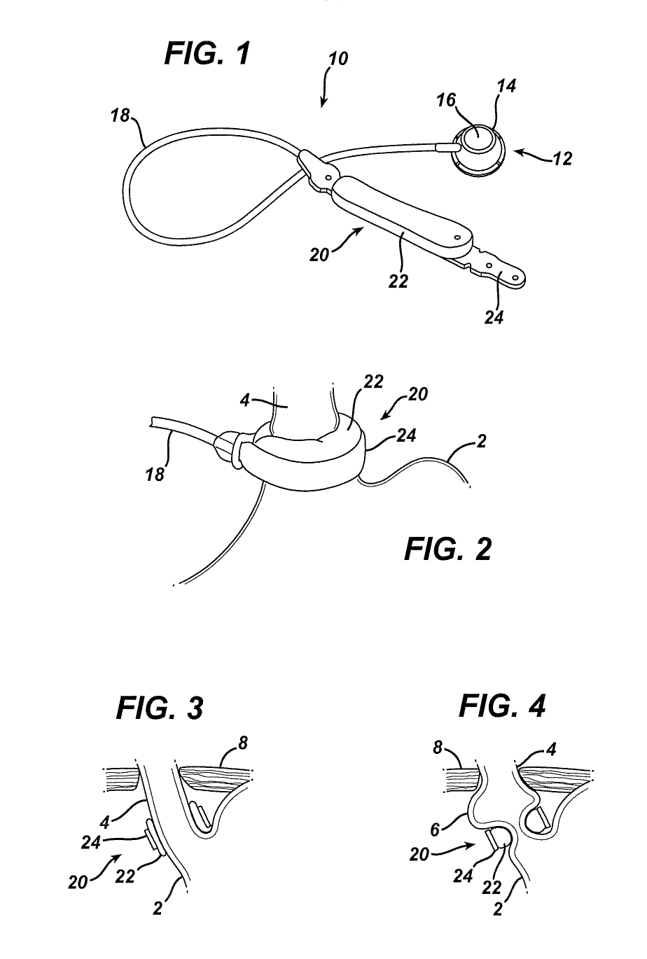

FIGS. 1-4 illustrate an exemplary gastric band system (10). As shown, gastric band system (10) comprises an injection port (12), a gastric band (20), and a catheter (18). Injection port (12) of the present example comprises a housing (14) and a needle penetrable septum (16). Housing (14) defines a fluid reservoir (not shown), such that a needle may pierce septum (16) to reach the reservoir and add or withdraw fluid (e.g., saline, etc.) as described in greater detail below. Housing (14) may be formed of titanium, plastic, or any other suitable material or combination of materials. Septum (16) may be formed of silicone or any other suitable material or combination of materials. Injection port (12) may be subcutaneously secured over a patient's sternum, to the patient's abdominal fascia, or in any other suitable location. In some versions, injection port (12) is configured and operable in accordance with the teachings of U.S. Pub. No. 2005/0283118, entitled “Implantable Medical Device with Simultaneous Attachment Mechanism and Method,” published Dec. 22, 2005, the disclosure of which is incorporated by reference herein. Alternatively, injection port (12) may have any other suitable configuration and/or operability.

Gastric band (20) of the present example comprises an inflatable bladder (22) that is secured to a flexible strap (24). Inflatable bladder (22) may be formed of silicone or any other suitable material or combination of materials. Catheter (18) provides fluid communication between bladder (22) and the reservoir of injection port (12). Accordingly, a needle that is inserted through septum (16) may be used to add or withdraw fluid from inflatable bladder (22), to adjust the restriction created by gastric band (20) as described in greater detail below. In some versions, gastric band (20) is configured and operable in accordance with the teachings of U.S. Pat. No. 7,416,528, entitled “Latching Device for Gastric Band,” issued Aug. 26, 2008, the disclosure of which is incorporated by reference herein. Alternatively, gastric band (20) may have any other suitable configuration and/or operability.

In some settings, gastric band (20) is applied about the gastro-esophageal junction of a patient. In particular, and as shown in FIG. 2, gastric band (20) is installed such that bladder (22) is adjacent to the tissue of the gastro-esophageal junction, with strap (24) on the outside of bladder (22). The ends of strap (24) are secured relative to each other when gastric band (20) is sufficiently wrapped about the patient's stomach (2). While strap (24) is flexible in this example, strap (24) substantially resists stretching along its length. Accordingly, when fluid is added to bladder (22) (e.g., using a needle inserted through septum (16) of injection port (12), etc.), bladder (22) expands and exerts inward forces on the gastro-esophageal junction of the patient. This reduces the size of the internal stoma at the gastro-esophageal junction, thereby creating a restriction on food intake into the patient's stomach (2). It should be understood that the size of this stoma may be decreased by adding more fluid to bladder (22) to create a greater degree of restriction; or increased by withdrawing fluid from bladder (22) to reduce the degree of restriction.

As shown in FIGS. 2-4, an installed gastric band (20) at least substantially encloses the upper portion of stomach (2) near the junction with esophagus (4) in the present example. FIG. 3 shows gastric band (20) in a deflated configuration, where bladder (22) contains little to no fluid, thereby maximizing the size of the stoma opening into stomach (2). FIG. 4 shows gastric band (20) in an inflated, fluid-filled configuration, where bladder (22) contains substantially more fluid than is shown in FIG. 3. In this configuration shown in FIG. 4, the pressure of gastric band (20) against stomach (2) is increased due to the fluid within bladder (22), thereby decreasing the stoma opening to create a food intake restriction. FIG. 4 also schematically illustrates the dilation of esophagus (4) above gastric band (20) to form an upper pouch (6) beneath the diaphragm muscle (8) of the patient.

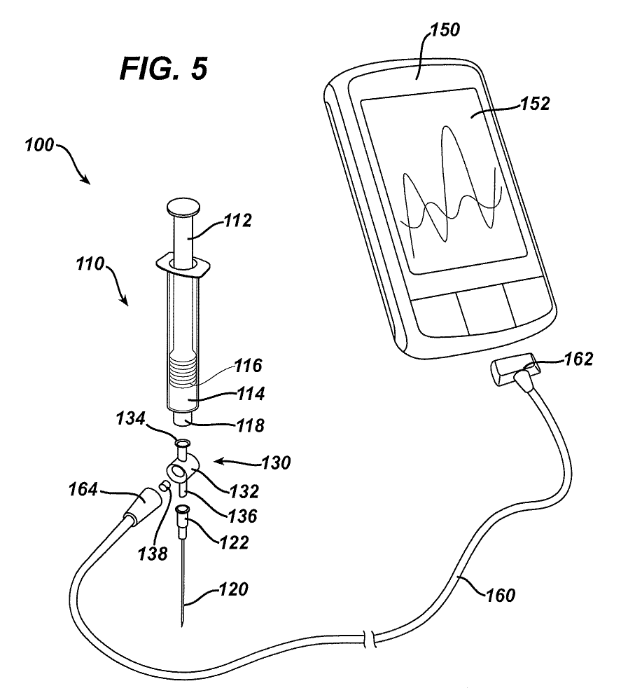

FIG. 5 shows an exemplary needle system (100) that may be used with gastric band system (10). In this example, needle system (100) comprises a syringe (110) and a display device (150) in communication via a cable (160). Syringe (110) comprises a plunger (112), a barrel (114), a pressure sensing component (130), and a needle (120). Plunger (112) includes a piston (116) that sealingly engages barrel (114). Barrel (114) includes a conventional luer lock portion (118) that is in fluid communication with the interior of barrel (114). Needle (120) comprises a conventional non-coring Huber needle, and includes a conventional luer lock portion (122). Of course, any of these components, among others, may be varied in any suitable fashion.

Pressure sensing component (130) of the present example comprises a body portion (132), an upper luer lock portion (134), a lower luer lock portion (136), and a pressure sensor (138). Upper luer lock portion (134) is configured to couple with luer lock portion (118) of syringe (110). Lower luer lock portion (136) is configured to couple with luer lock portion (122) of needle (120). It should therefore be understood that pressure sensing component (130) of the present example may be retrofitted to a variety of types of syringes and needles, etc. Body portion (132) provides communication of fluid between syringe (110) and needle (120). In addition, pressure sensor (138) is in fluid communication with the interior of body portion (132), such that pressure sensor (138) is operable to sense the pressure of fluid in syringe (110) and needle (120) as will be described in greater detail below. In some versions, pressure sensor (138) comprises a pressure sensor provided by CardioMEMS, Inc. of Atlanta, Ga., or OpSens of Quebec, Canada, though any other suitable type of pressure sensor may be obtained from any other suitable source. By way of example only, pressure sensor (138) may be constructed in accordance with the teachings of U.S. Pat. No. 6,855,115, entitled “Implantable Wireless Sensor for Pressure Measurement within the Heart,” issued Feb. 15, 2005, the disclosure of which is incorporated by reference herein. As another merely illustrative example, pressure sensor (138) may be constructed in accordance with the teachings of U.S. Pub. No. 2006/0211914, entitled “System and Method for Determining Implanted Device Positioning and Obtaining Pressure Data,” published Sep. 21, 2006, the disclosure of which is incorporated by reference herein. Still other suitable forms pressure sensor (138) may take will be apparent to those of ordinary skill in the art in view of the teachings herein.

Cable (160) of the present example has a boot portion (164), which is configured to selectively attach to pressure sensing component (130). Boot portion (164) includes a feature (not shown) that is operable to electrically engage with pressure sensor (138) and thereby communicate pressure readings obtained by pressure sensor (138) along cable (160). Such a feature may comprise one or more terminals (not shown) or any other suitable type(s) of feature(s) as will be apparent to those of ordinary skill in the art. In the present example, boot portion (164) is removeably coupled with pressure sensing component (130), though it should be understood that such a coupling may be substantially permanent or integral, etc. The other end of cable (160) includes a connector (162) that couples with display device (150). Cable (160) is thereby operable to communicate data obtained by pressure sensor (138) to display device (150). Display device (150) is operable to process such data and render feedback to the user via display (152).

In some versions, display device (150) comprises a dedicated device constructed for the purpose of processing pressure data and providing graphical and/or textual output to the user via display (152). In some other versions, display device (150) comprises a conventional portable electronic device (e.g., a BlackBerry, an iPhone, a laptop computer, etc.) with software that is operable to process pressure data and provide graphical and/or textual output to the user. In still other versions, display device (150) comprises a desktop PC or other type of computer with software that is operable to process pressure data and provide graphical and/or textual output to the user. Still various other forms that display device (150) may take will be apparent to those of ordinary skill in the art in view of the teachings herein. It should also be understood that cable (160) may comprise a conventional USB cable or some other type of cable. Furthermore, cable (160) may be omitted in some versions, such as versions where pressure sensing component (130) is operable to communicate to display device (150) wirelessly. Examples of such wireless communication are disclosed in U.S. Pub. No. 2006/0211914, entitled “System and Method for Determining Implanted Device Positioning and Obtaining Pressure Data,” published Sep. 21, 2006, the disclosure of which is incorporated by reference herein; while other examples of wireless communication will be apparent to those of ordinary skill in the art in view of the teachings herein.

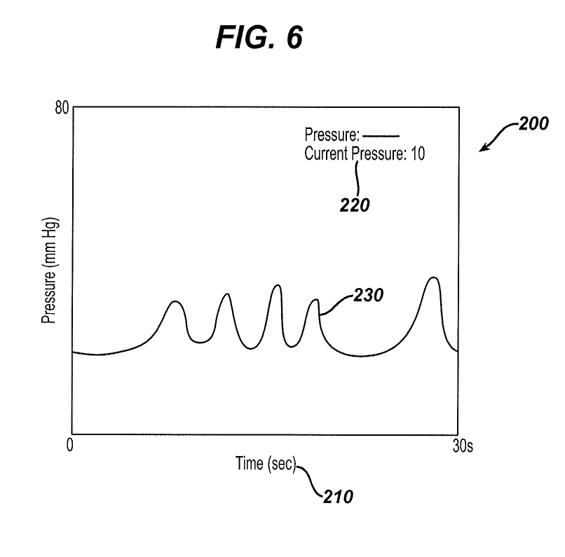

Display (152) may be used to provide a graphical user interface as shown in FIG. 6. Display (152) includes a plot or graph (200) of pressure over time, which is shown as a line graph (230) but could also be shown as a bar graph, scatter graph, or virtually any other graphic representation. The time scale along the horizontal axis (210) can be automatically sized to the amount of pressure data available or can be user-adjustable, e.g., to examine a time period of interest. Display (152) can also include a textual indicator (220), which numerically provides a current or instantaneous pressure reading. A wide variety of other kinds of information also can be presented on display (152), including a baseline indicator (not shown) showing a steady-state or baseline value of the pressure; and pulse indicators (not shown) showing the number of pulses (e.g., the pulses may be pressure pulses which can represent or be caused by the peristaltic contractions of a patient swallowing). Display (152) can include pressure readings taken from prior visits (e.g., prior visits of the same patient, or from previous adjustments of the restriction device), and/or pressure readings of previous peristaltic events representing swallowing, heart rate, breathing rate, or virtually any other physiological parameter. Display (152) also can include a patient's name or other identifying information, along with notes, lists of activities or guidelines for the patient, and so on. Display (152) and accompanying graphical user interface may be further configured in accordance with the teachings of U.S. Pub. No. 2008/0250340, entitled “GUI for an Implantable Restriction Device and a Data Logger,” published Oct. 9, 2008, the disclosure of which is incorporated by reference herein.

In one merely exemplary use, where gastric band system (10) has been implanted in a patient, needle (120) is inserted into a patient to reach septum (16) of injection port (12). Upon such insertion, needle (120) is in fluid communication with gastric band system (10), such that the pressure of the fluid in gastric band system (10) and needle system (100) will be substantially equalized. It will therefore be appreciated that fluid pressure sensed by pressure sensor (138) may be indicative of the pressure of fluid within gastric band system (10). In some settings, such pressure information may be useful during a process of using needle system (100) to adjust fluid pressure of gastric band system (10) by adding or withdrawing fluid to or from gastric band system (10). In particular, the configuration of syringe (110) and pressure sensing component (130) may permit substantially simultaneous adjustment and reading of fluid pressure.

For instance, a user may first insert needle (120) into the patient to reach septum (16) of injection port (12). Upon pressure equalization, the user may then read the initial pressure via display device (150). It will be understood that pressure equalization may be determined by a pressure reading remaining substantially constant. The user may then add or withdraw fluid to or from gastric band system (10) using syringe; by pushing plunger (112) further into barrel (114) or withdrawing plunger (112) further from barrel (114), respectively. The user may monitor display device (150) during such adding/withdrawing of fluid to monitor the fluid pressure in substantially real time. To the extent that there is a delay between the user's manipulation of syringe (110) and the time the pressure substantially equalizes among syringe (110) and gastric band system (10), the user may simply wait until the pressure reading indicated through display device (150) becomes substantially constant. Still other suitable ways in which needle system (100) may be used in conjunction with a gastric band system (10) will be apparent to those of ordinary skill in the art in view of the teachings herein.

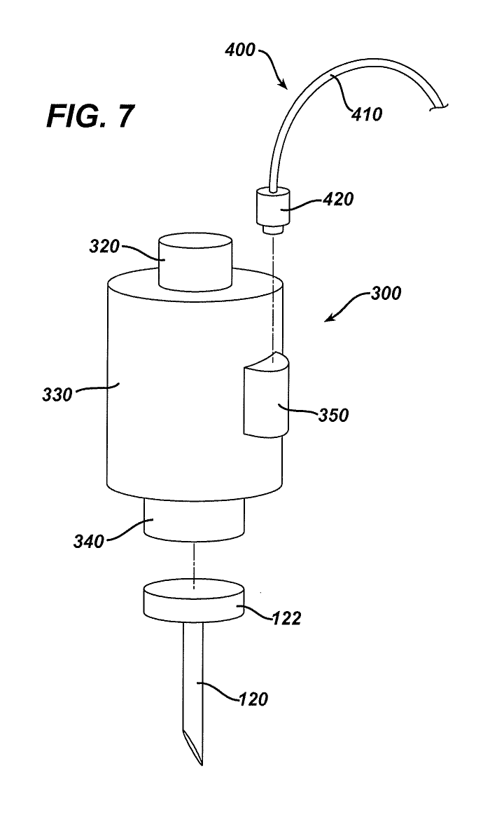

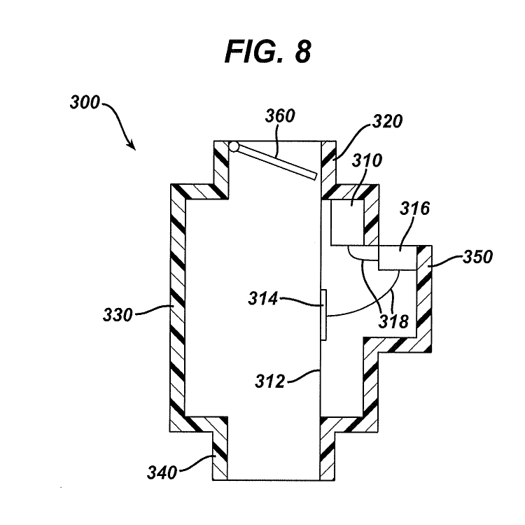

Turning now to FIGS. 7-8, FIG. 7 depicts a version of pressure sensing component (300) for use with an accelerometer (310). Pressure sensing component (300) comprises an upper luer lock portion (320), a fluid housing portion (330), a lower luer lock portion (340), and a cable interface portion (350). Pressure sensing component (300) may be removeably coupled with reusable connector (400). Reusable connector (400) comprises a cable (410) and an interface portion (420). Pressure sensing component (300) may be combined with a syringe (110) and a display device (150), as a substitute for pressure sensing component (130) described above.

Upper luer lock portion (320) is in fluid communication with the hollow interior of fluid housing portion (330), and is configured to releaseably couple with luer lock portion (118) of syringe (110) to allow fluid communication between syringe (110) and fluid housing portion (330). Other suitable ways may be used to provide fluid communication between fluid housing portion (330) and syringe (110), as will be apparent to one of ordinary skill in the art. Lower luer lock portion (340) is also in fluid communication with the hollow interior of fluid housing portion (330), and is configured to releasably couple with luer lock portion (122) of needle (120) to allow fluid communication between needle (120) and fluid housing portion (330). Alternatively, fluid housing portion (330) and needle (120) may be in fluid communication in any other suitable fashion. It should be understood that, when needle (120) is coupled with lower luer lock portion (340) and when needle (120) is inserted into septum (16) of injection port (12), the interior of fluid housing portion (330) is also in fluid communication with gastric band system (10) in the present example.

As shown in FIG. 8, upper luer lock portion (320) further comprises a check valve (360) configured to only allow one-way flow of a fluid from syringe (110) to pressure sensing component (300). Check valve (360) has an open position and closed position. Check valve (360) may be positioned and configured such that check valve (360) is in the open position when luer lock portion (118) of syringe (110) is coupled with luer lock portion (320) of pressure sensing component (300); and such that check valve (360) is in the closed position when luer lock portion (118) of syringe (110) is not coupled with luer lock portion (320) of pressure sensing component (300). In the open position, fluid may freely pass through check valve (360), in either direction (e.g., from pressure sensing component (300) into syringe (110) and from syringe (110) into pressure sensing component (300), etc.). In the closed position, check valve (360) creates a fluid-tight seal to prevent the fluid flow back to syringe (110) or any other fluid leakage from pressure sensing component (300). By preventing fluid flow from fluid housing portion (330) to syringe (110) with check valve (360), syringe (110) may be removed from upper luer lock portion (320) after plunger (112) has been engaged to transfer fluid from syringe (110) to gastric band (20) through septum (16) of injection port (12), without fluid escaping fluid housing portion (330). Of course, as with other components described herein, check valve (360) is merely optional. By way of example only, a conventional stopcock or other type of valve may be positioned between syringe (110) and pressure sensing component (300), if desired.

Upper luer lock portion (320) is in fluid connection with fluid housing portion (330) and may be integrally formed with fluid housing portion (330), cable interface portion (350), and/or lower luer lock portion (340). Alternatively, upper luer lock portion (320) may be constructed separately from fluid housing portion (330), cable interface portion (350), and/or lower luer lock portion (340) and thereafter assembled together using fluid tight seals or by any means suitable as would be apparent to one of ordinary skill in the art in view of teachings herein.

Returning now to FIG. 7, fluid housing portion (330) is in fluid communication with upper luer lock portion (320) and lower luer lock portion (340). Further, fluid housing portion (330) is in communication with cable interface portion (350). Fluid housing portion (330) is generally hollow as shown in FIG. 8, and contains a diaphragm (312) and a strain gauge (314). Diaphragm (312) is configured to flex in response to the pressure of fluid within the interior of fluid housing portion (330). A strain gauge (314) is positioned on the opposite side of diaphragm (312). Strain gauge (314) is configured to detect flexing and/or stretching strain of diaphragm (312), such that strain gauge (314) can sense the pressure of fluid within the interior of fluid housing portion (330) as a function of flexing of diaphragm (312). Strain gauge (314) is in electrical communication with an electrical contact (316) in cable interface portion (350), which allows strain gauge (314) to communicate pressure data associated with the pressure of fluid within fluid housing portion (330). Diaphragm (312) and strain gauge (314) thus together provide a pressure sensor that is similar to a diaphragm-based sensor as described in U.S. Pub. No. 2006/0211914, entitled “System and Method for Determining Implanted Device Positioning and Obtaining Pressure Data,” published Sep. 21, 2006, the disclosure of which is incorporated by reference herein. Of course, any other suitable type of pressure sensor may be used. By way of example only, fluid housing portion (330) may be easily modified to incorporate any of the pressure sensing components, features, and/or configurations disclosed in U.S. Pub. No. 2006/0211914.

Further, diaphragm (312) hermetically seals strain gauge (314) and wires (318) from fluid that is within the hollow interior of fluid housing portion (330), such that strain gauge (314), accelerometer (310), wires (318), and cable interface portion (350) remain substantially dry during use of pressure sensing component (300), even as pressurized fluid resides within the hollow interior of fluid housing portion (330).

Fluid housing portion (330) also comprises accelerometer (310) located within the interior of fluid housing portion (330). Accelerometer (310) is configured to sense orientational changes of pressure sensing component (300) relative to gravity (e.g., tilt angle, etc.), such that data associated with the output of accelerometer (310) can be used to determine whether pressure sensing component (300) is in an upright position (e.g., substantially perpendicular to the surface of the ground), a sideways position (e.g., substantially parallel to the surface of the ground), an upside-down position, and variations and orientations in between those positions. Accelerometer (310) may thus be used to measure both dynamic and static measurements of acceleration. Acceleration data may correlate to the direction and velocity of movement in a dynamic measurement. It should be understood that such data indicating the orientation/movement of pressure sensing component (300) may be indicative of the orientation/movement of the patient and components of gastric band system (10) implanted in the patient.

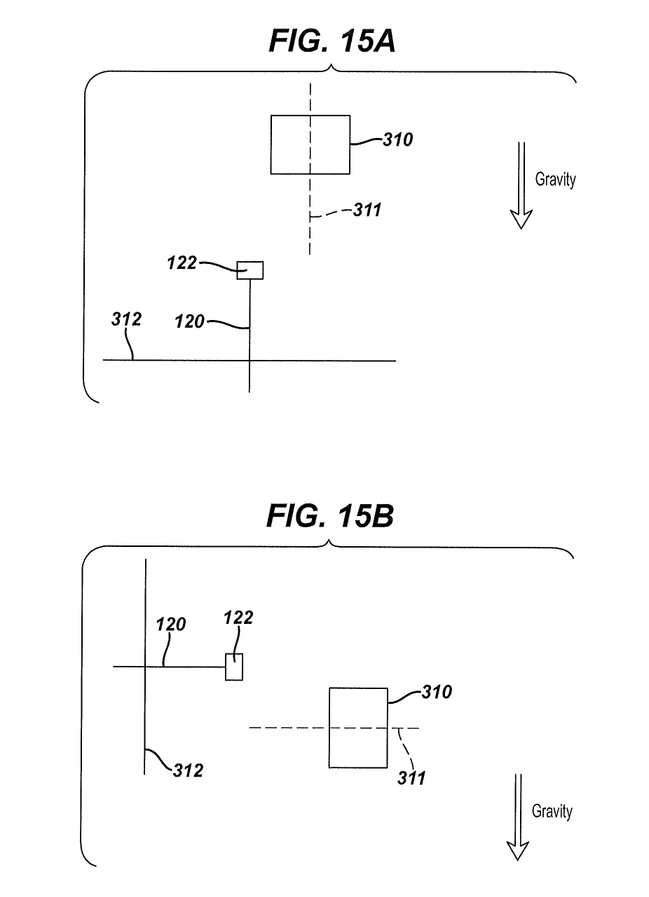

By way of example only, and as shown in FIGS. 15A-15B, accelerometer (310) may define a reference z-axis (311) that is substantially parallel to an axis defined by needle (120). Accelerometer (310) may realize and report the earth's gravity as “+1 g.” The orientation of accelerometer (310) may be determined by comparing a gravitational signal obtained through accelerometer (310) to “+1 g.” In some such versions, accelerometer (310) may provide an output indicative of a “+1 g” acceleration/orientation when needle (120) is inserted in a patient's abdomen (312) when the patient is lying on their back, as shown in FIG. 15A. In such a position, the reference z-axis (311) and needle (120) may be substantially parallel to the vertical direction in which gravity is directed. Similarly, in some such versions, accelerometer (310) may provide an output indicative of a “0 g” acceleration/orientation when needle (120) is inserted in a patient's abdomen (312) when the patient is in a sitting or standing position, as shown in FIG. 15B. In such a position, the reference z-axis (311) and needle (120) may be substantially perpendicular to the vertical direction in which gravity is directed. Accelerometer (310) may provide outputs of different factors of “g” when the patient is at orientational positions between lying down on their back and sitting/standing. In other words, the factor of “g” may be a function of the angle defined between the reference z-axis (311) of accelerometer (310) and the vertical axis along which gravity operates. For instance, the factor of “g” may be equal to the cosine of such an angle. Of course, as noted elsewhere herein, accelerometer (310) may have several reference axes in some versions. It should also be understood that the sensitivity of accelerometer (310) may or may not vary based on the orientation of accelerometer (310).

Furthermore, accelerometer (310) may be able to sense various other physiological parameters of the patient, including but not limited to swallowing, heart rate, breathing rate, etc. For instance, such physical parameters may be communicated as movement through inserted needle (120) to accelerometer (310). Accelerometer (310) may be of any suitable type as would be apparent to one of ordinary skill in the art in view of the teachings herein, including but not limited to the following types: a piezoelectric accelerometer, a strain gauge-based accelerometer, a capacitive accelerometer, a piezoresistive accelerometer, a hall effect accelerometer, a magnetoresistive accelerometer, a heat transfer accelerometer, etc. Accelerometer (310) may comprise a multi-axis accelerometer (not shown) or a plurality of accelerometers (not shown). Further, accelerometer (310) may be powered by power traveling through cable (410) from an external such that no local source of power (e.g., battery and/or capacitor within pressure sensing component (300), etc.) is necessarily needed. Alternatively, a separate battery and/or capacitor, etc. may be used to power accelerometer (310).

Data from accelerometer (310) is transmitted via wire (318) to electrical contact (316) in cable interface portion (350) in the present example. Accelerometer (310) may be removeably or fixedly attached to the interior of fluid housing portion (330). In alternative versions (not shown), accelerometer (310) may be located anywhere within the interior of pressure sensing component (300) or may be located on the external surface of pressure sensing component (300) as would be apparent to one of ordinary skill in the art in view of the teachings herein. Further, accelerometer (310) may be located separately from pressure sensing component (300) as will be described in greater detail below.

As discussed above, accelerometer (310) is operably configured to sense the orientation of pressure sensing component (300) and/or other physiological parameters of the patient. As also noted above, pressure sensing component (300) may be releaseably attached to syringe (110) and also releaseably attached to needle (120). Thus, the orientation of the pressure sensing component (300) may be indicative of orientation of the syringe (110) and needle (120). Further, when needle (120) is inserted into septum (16) of injection port (12), needle (120) and septum (16) (and tissue surrounding the inserted portion of needle (120)) form a substantially rigid connection. In this sense, movement by the patient (e.g., fidgeting, sitting up, lying down, heart rate, breathing, swallowing, etc.) may cause a corresponding movement in needle (120), which is releaseably attached to pressure sensing component (300), wherein the pressure sensing component (300) contains accelerometer (310). Thus, movements, changes in orientation by the patient, and/or other physiological parameters of the patient are detectable by accelerometer (310) through corresponding movements of needle (120) and pressure sensing component (300). Additionally, in alternative versions (not shown) accelerometer (310) may be substituted or supplemented with a gravitometer (not shown) or may be used in conjunction with a gravitometer (not shown) to provide further information regarding positioning, movement, and/or orientation of the patient. Accelerometer (310) may be configured to have adjustable sensitivity settings such that minimal movements in a patient (e.g., fidgeting, coughing, burping, hiccupping, talking, etc.) are detectable or non-detectable. Alternatively, a processor in communication with accelerometer (310) that processes data from accelerometer (310) (e.g., a processor in display device (150), etc.) may be configured to filter or essentially ignore data indicating negligible movement sensed by accelerometer (310); or to react to even minute movement sensed by accelerometer (310).

As shown in FIG. 8, cable interface portion (350) comprises electrical contact (316). Electrical contact (316) is in communication with accelerometer (310) and strain gauge (314) via respective wires (318). Returning to FIG. 7, electrical contact (316) is located in cable interface portion (350) of pressure sensing component (300) such that when interface portion (420) of reusable connector (400) is removeably coupled with c able interface portion (350), an electrical connection is made between interface portion (420) of reusable connector (400) and electrical contact (316) in pressure sensing component (300). Thus, when in communication with electrical contact (316), reusable connector (400) is operable to transmit accelerometer data and pressure data to display device (150) via cable (410). Interface portion (420) of connector (400) and interface portion (350) of pressure sensing component (300) may also include complementary features that resist decoupling of interface portions (350, 420) after interface portions (350, 420) have been coupled. By way of example only, interface portions (350, 420) may include features to provide a screw-in fitting, a bayonet fitting, a snap fitting, an interference fitting, or any other suitable type of fitting.

Of course, reusable connector (400) may instead be substituted or supplemented with a variety of other types of communication devices, including wireless communication devices (e.g., infrared transmitter, RF transmitter, etc.). Various suitable alternative communication devices are disclosed in U.S. Pub. No. 2006/0211914, entitled “System and Method for Determining Implanted Device Positioning and Obtaining Pressure Data,” published Sep. 21, 2006, the disclosure of which is incorporated by reference herein. Other suitable types of alternative communication devices will be apparent to those of ordinary skill in the art in view of the teachings herein.

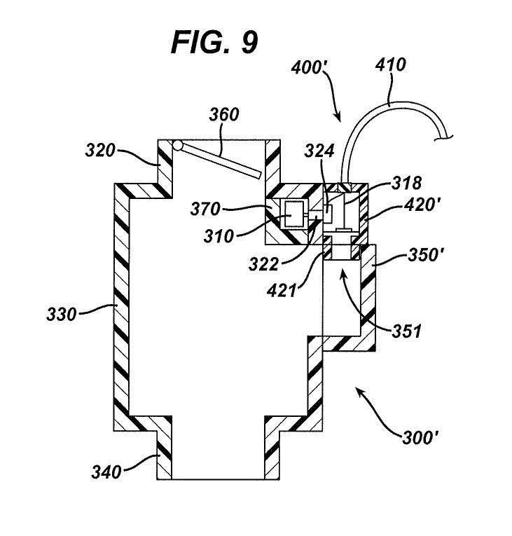

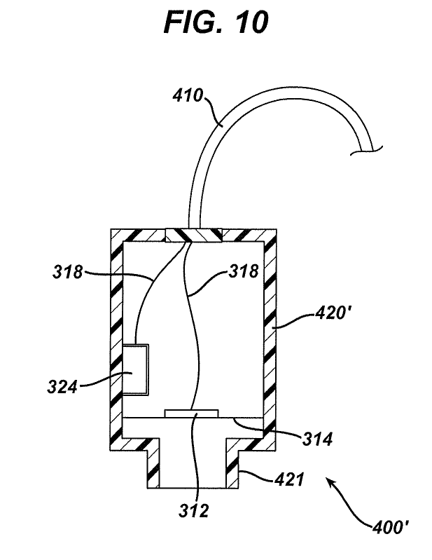

Turning now to FIGS. 9-10, FIG. 9 depicts an alternative version of pressure sensing component (300′) wherein diaphragm (312) and strain gauge (314) are instead located within an alternative version of reusable connector (400′). Pressure sensing component (300′), which comprises upper luer lock portion (320), fluid housing portion (330), cable interface portion (350′), and lower luer lock portion (340), may be constructed of disposable materials such that pressure sensing component (300′) is disposable and may be replaced prior to each use.

In the example shown in FIGS. 9-10, accelerometer (310) is located in a hermetically sealed portion (370) of pressure sensing component (300′). Accelerometer (310) is in electrical communication with a side electrical contact (322) configured to allow accelerometer (310) to communicate measurements and/or electrical signals from interior of pressure sensing component (300) to exterior of pressure sensing component (300′). As shown in FIG. 10, reusable connector (400′) contains an internal electrical contact (324) in electrical communication with exterior of interface portion (420′) of reusable connector (400). Internal electrical contact (324) is in electrical communication with cable (410), which is configured to communicate signals to display (150). Thus, when reusable connector (400′) couples with cable interface portion (350′) of pressure sensing component (300′), internal electrical contact (324) and side electrical contact (322) form an electrical connection whereby accelerometer (310) is in electrical communication with display (150). When reusable connector (400′) decouples from cable interface portion (350′), internal electrical contact (324) and side electrical contact (322) cease to be in electrical communication.

Returning to FIG. 9, fluid housing portion (330) is in fluid communication with cable interface portion (350′). Cable interface portion (350′) further includes a port (351) that provides a path for fluid communication from the interior of fluid housing portion (330). Port (351) is configured to insertingly receive a port (421) of interface portion (420′). In particular, port (421) is configured to provide a fluid coupling between cable interface portion (350′) and interface portion (420′). This coupling is also configured to be sealed to prevent fluid leakage at the interface of ports (351, 421).

It should therefore be understood that, upon coupling of cable interface portion (350′) with interface portion (420′) of reusable connector (400′), cable interface portion (350′) is also in fluid communication with the interior of interface portion (420′). As stated above diaphragm (312) and strain gauge (314) are positioned in interface portion (420′) of reusable connector (400′). Diaphragm (312) hermetically seals strain gauge (314), side electrical contact (322), and wires (318) from fluid that is within the interface portion (420′) of reusable connector (400′). As a result of fluid housing portion (330) being in fluid communication with cable interface portion (350′), when needle (120) is inserted in septum (16) of injection port (12), cable interface portion (350′) is in fluid communication with gastric band system (10). The fluid pressure in the interior of cable interface portion (350′) is substantially equalized with fluid pressure in gastric band system (10). In the event that fluid pressure in the interior of cable interface portion (350′) and gastric band system (10) are not substantially equalized, the cable interface portion (350′) and gastric band system (10) may remain in fluid communication until their respective internal fluid pressures are substantially equalized. In some settings, substantial equalization of fluid pressures may be presumed when the pressure data obtained from pressure sensing component (300, 300′) remains substantially constant.

Fluid pressure in cable interface portion (350) may be measured as stated above by using diaphragm (312) and strain gauge (314) in such a way that strain gauge (314) detects diaphragm (312) flexing in response to fluid pressure in cable interface portion (350). Strain gauge (314) may then communicate the signal associated with diaphragm (312) flexing in response to fluid pressure in cable interface portion (350) to display (150) through cable (410).

It should also be understood that, as with accelerometer (310) in pressure sensing component (300) of FIGS. 7-8, accelerometer (310) in pressure sensing component (300′) of FIG. 9 may still be used to detect the orientation of the patient, movement by the patient, and/or other physiological parameters of the patient (e.g., fidgeting, sitting up, lying down, heart rate, breathing, swallowing, etc.) when needle (120) is inserted in the patient. Such data may be used when gathered from accelerometer (310) in pressure sensing component (300′) of FIG. 9 in the same manner described above with respect to data gathered from accelerometer (310) in pressure sensing component (300) of FIGS. 7-8. Alternatively, data gathered from accelerometer (310) in either version of pressure sensing component (300, 300′) may be used in any other suitable fashion, as will be apparent to those of ordinary skill in the art in view of the teachings herein.

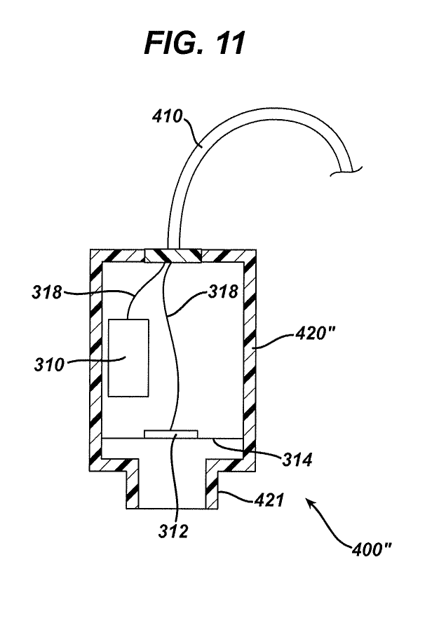

Turning now to FIG. 11, FIG. 11 depicts another alternative version of reusable connector (400″), wherein accelerometer (310), diaphragm (312), and strain gauge (314) are located in the interior of interface portion (420″) of reusable connector (400″). Both accelerometer (310) and strain gauge (314) are in electrical communication with cable (410) via wires (318), whereby electrical signals may be communicated through cable (410) to display (150) to process such data and render feedback to the user via display (152).

Interface portion (420″) of reusable connector (400″) is configured to detachably couple with a cable interface portion of an alternative pressure sensing component (not shown). In some versions, such an alternative pressure sensing component is substantially identical to pressure sensing component (300′) shown in FIG. 9 and described above, except that this alternative pressure sensing component lacks accelerometer (310) since accelerometer (310) is provided in interface portion (420″) of reusable connector (400″). Alternatively, accelerometers (310) may be provided in both reusable connector (400″) and pressure sensing component (300′), if desired. In the present example, when interface portion (420″) couples with the cable interface portion of the alternative pressure sensing component, the interior of interface portion (420″) is in fluid connection with interior of the alternative pressure sensing component in such a way that their respective interior fluid pressures are substantially equalized. If the alternative pressure sensing component is releaseably attached to needle (120) and needle (120) is inserted in septum (16) of injection port (12), then gastric band system (10) and interior of interface portion (420″) would have substantially equalized fluid pressures. Thus, the fluid pressure of gastric band system (10) may be measured by using strain gauge (314) with diaphragm (312). Diaphragm (312) also hermetically seals strain gauge (314) accelerometer (310), and wires (318) from fluid within interface portion (420″) of reusable connector (400″).

It should also be understood that, as with accelerometers (310) in pressure sensing components (300, 300′) of FIGS. 7-9, accelerometer (310) in interface portion (420″) of reusable connector (400″) of FIG. 11 may still be used to detect the orientation of the patient, movement by the patient, and/or other physiological parameters of the patient (e.g., fidgeting, sitting up, lying down, heart rate, breathing, swallowing, etc.) when needle (120) is inserted in the patient. Such data may be used when gathered from accelerometer (310) in interface portion (420″) of reusable connector (400″) of FIG. 11 in the same manner described above with respect to data gathered from accelerometer (310) in pressure sensing component (300) of FIGS. 7-8. Alternatively, data gathered from accelerometer (310) in either version of pressure sensing component (300, 300′) or in interface portion (420″) of reusable connector (400″) may be used in any other suitable fashion, as will be apparent to those of ordinary skill in the art in view of the teachings herein.

In some alternative versions (not shown), it will be appreciated that accelerometer (310) need not be placed only in interface portion (420″) or only in the interior of fluid housing portion (330) of pressure sensing component (300, 300′). Accelerometer (310) may be placed anywhere that one of ordinary skill in the art would find suitable in view of the teachings herein, such as, for example, in syringe (110), in plunger (112), or in needle (120). Additionally, accelerometer (310) may be substituted with a gravitometer (not shown), multiple accelerometers (not shown), or a multi-axis accelerometer (not shown). Still other suitable variations of, substitutes for, supplements for, configurations of, and locations for accelerometer (310) will be apparent to those of ordinary skill in the art in view of the teachings herein.

The following description will refer back to the example of pressure sensing component (300) described above with reference to FIGS. 7-8, though it should be understood that the following description may be equally applicable to the illustrative variations described above with respect to FIGS. 9-11. As described above, upper luer lock portion (320) of pressure sensing component (300) may be releaseably attached to syringe (110) and lower luer lock portion (340) may be releaseably attached to needle (120) so as to provide fluid communication from syringe (110) to needle (120). Upon placing needle (120) in septum (16) of injection port (12), the user may push or pull plunger (112) to add or withdraw fluid from gastric band system (10). The user may monitor display device (150) during such adding/withdrawing of fluid to monitor the fluid pressure in substantially real time. It will be appreciated that movements by the patient may affect the pressure readings associated with gastric band system (10). For example, a patient that moves from a supine position to a seated position may cause the pressure of the gastric band system (10) to temporarily spike such that the measured pressure as seen on display device (150) will include pressure changes resulting from patient movement. Even small movements, such as those associated fidgeting, coughing, burping, hiccupping, talking, etc. may affect the measured pressure as seen on display device (150). It will be appreciated that added “noise” caused by patient's movement may be generally unhelpful for determining whether the intraband pressure in gastric band system (10) is at a suitable level. Thus, understanding whether a patient has moved may be useful to determine relative significance of measured and/or displayed pressure data.

In one merely exemplary use of pressure sensing component (300) with accelerometer (310), gastric band system (10) is implanted in a patient. Needle (120) is connected to pressure sensing component (300), which contains accelerometer (310), and pressure sensing component (300) is connected to syringe (110). Needle (120) is inserted into a patient to reach septum (16) of injection port (12). Upon insertion of needle (120) through septum (16), needle (120) is in fluid communication with gastric band system (10), such that the pressure of the fluid in gastric band system (10) and needle system (100) will eventually be substantially equalized. Inserted needle (120) also forms a substantially rigid connection with septum (16) of injection port (12), such that movement of the patient and other physiological parameters of the patient may be communicated through needle (120) to pressure sensing component (300) and accelerometer (310). It will be appreciated that a substantial amount of movement at and/or around accelerometer (310) may be indicative of movement at and/or around gastric band (20). It will be further appreciated that movement at and/or around gastric band (20) may cause changes in pressure of fluid within gastric band system (10). Similarly, the orientation of pressure sensing component (300) and accelerometer (310) may be indicative of the orientation of the patient. In some settings, information on the pressure of fluid within gastric band system (10) may be useful during a process of using needle system (100) to adjust fluid pressure of gastric band system (10) by adding or withdrawing fluid to or from gastric band system (10). However, it may be desirable in some situations to account for pressure changes caused by patient movement and/or other physiological parameters of the patient when evaluating data showing pressure of fluid in gastric band system (10) to determine what kind of adjustment needs to be made (if any) to the fluid pressure. In particular, it may be desirable in some situations to ignore pressure changes caused by certain types of patient movement, to filter out pressure data associated with certain types of patient movement or other activities by the patient, and/or to dynamically compensate for certain physiological parameters of the patient (e.g., fidgeting, sitting up, lying down, heart rate, breathing, swallowing, other parameters that might impact the fluid pressure, etc.) when processing pressure data.

For instance, a user may first insert needle (120) into the patient laying in a supine position to reach septum (16) of injection port (12). Upon pressure equalization, the user may then read the initial pressure and accelerometer data via display device (150). The user may then remove syringe (110) from patient (e.g., when a valve is available to cut off fluid communication through luer lock portion (320), etc.), leaving needle (120) and pressure sensing component (300) connected to the patient. If desired, the user may also tape or otherwise brace needle (120) and pressure sensing component (300) to the patient. Pressure readings and accelerometer data are continually updated to display device (150), even with syringe (110) removed. Alternatively, the user may leave syringe (110) coupled with pressure sensing component (300), leaving the whole assembly of syringe (110), pressure sensing component (300), and needle (120) connected with the patient as pressure readings and accelerometer data are continually updated. The user may then ask the patient to sit up from the supine position. Sitting up by the patient may cause a temporary increase in the pressure reading. The user may then verify that a corresponding change in the accelerometer reading occurred by the patient sitting up. For instance, display device (150) may annotate the pressure reading or otherwise account for the change in the accelerometer reading when displaying pressure data, as described in greater detail below. The user may thereafter ignore, or reduce the significance of, pressure increases or decreases accompanied by a corresponding change in accelerometer data.

After determining whether the gastric band system (10) should have additional fluid inserted or removed, syringe (110) may be re-coupled with pressure sensing component (300) to perform the fluid adjustment. As the user adds/removes fluid, the user may monitor the fluid pressure in real time, which includes appropriately weighing the significance of fluid pressure spikes or other changes that are accompanied by corresponding, or substantially corresponding, changes in accelerometer data. The user may monitor display device (150) during such adding/withdrawing of fluid to monitor the fluid pressure in substantially real time. To the extent that there is a delay between the user's manipulation of syringe (110) and the time the pressure substantially equalizes among syringe (110) and gastric band system (10), the user may simply wait until the pressure reading indicated through display device (150) becomes substantially constant. Still other suitable ways in which needle system (100) may be used in conjunction with a gastric band system (10) will be apparent to those of ordinary skill in the art in view of the teachings herein.

The following examples relate to various types of graphs (500, 500′, 500″) that may be provided through display (152) of display device (150). As described in greater detail below, these graphs (500, 500′, 500″) relate to pressure data obtained from pressure sensing component (200, 300, 300′) and data obtained from accelerometer (310) to a display device (150). It should be understood that any of these graphs (500, 500′, 500″) may be readily incorporated into the various types of graphical user interfaces taught by U.S. Pub. No. 2008/0250340, entitled “GUI for an Implantable Restriction Device and a Data Logger,” published Oct. 9, 2008, the disclosure of which is incorporated by reference herein. Alternatively, any of these graphs (500, 500′, 500″) may be incorporated into any other suitable type of display.

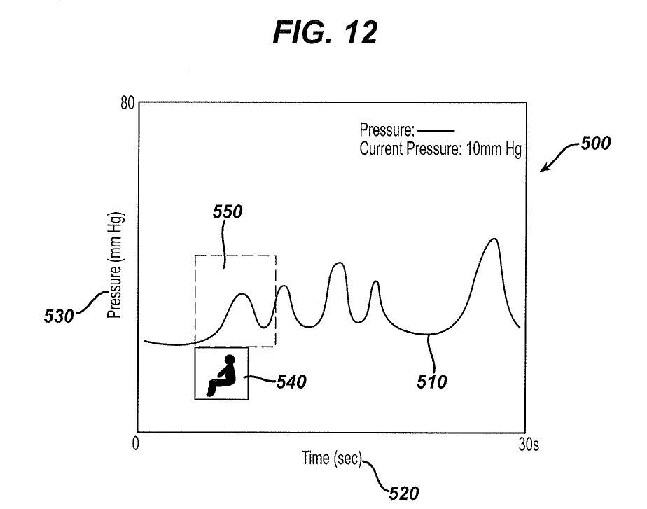

Turning now to FIG. 12, an exemplary graph (500) is shown, which may be part of a graphical user interface provided through display (152) of display device (150). Graph (500) shows a line graph (510) of pressure data from gastric band system (10). Such pressure data may be obtained using any type of pressure sensing component (200, 300, 300′) described herein, including variations thereof. Time (520) is represented by the horizontal axis, and is measured in seconds, but may be measured in any other suitable unit of measurement. Pressure (530) is represented by vertical axis and measured in mmHG, but may use any other suitable unit of measurement. Line graph (510) is plotted real time or near-real time in the present example (e.g., line graph (510) is plotted as fluid pressure is sensed by pressure sensing component (200, 300, 300′)), though it should be understood that any suitable delay may be provided. While recording and plotting pressure data, accelerometer data from accelerometer (310) is also recorded and/or updated. Changes in accelerometer data that may be indicative of the patient sitting up or otherwise moving or changing position, etc., are thus recognized by display device (150).

Accelerometer data indicative of a patient sitting up may be represented by an icon (540) of an illustrated person sitting up. Icon (540) may thus be used as a time stamp to annotate pressure readings, with such annotations being based on data from accelerometer (310). Other icons may include (but need not be limited to) graphical representations of a person lying down or standing up, coughing, etc. Further, a portion of graph (500) corresponding to the patient sitting up may be highlighted (550). As a result, the user may view graph (500) and see that a particular change in pressure was caused by or otherwise related to the patient sitting up as indicated by icon (540) and as indicated by the associated highlighted portion (550). In other words, software in the display device (150) may automatically annotate pressure readings on graph (500) with icons or other indicia showing how some pressure readings are associated with movement of the patient, changes in patient positioning, changing in other physiological parameters of the patient, etc., as detected by accelerometer (310). Thus, the user and/or software may ignore or factor out what might otherwise appear to be relevant pressure data because the change in pressure was in fact caused by the patient's movement or positioning, etc. This annotation of pressure data may allow the user to focus on other, more pertinent readings of fluid pressure or changes in fluid pressure of gastric band system (10). Such annotations are provided in real time or near-real time in the present example (e.g., icon (540) is placed on graph (500) as movement/orientation is sensed by accelerometer (310)), though it should be understood that any suitable delay may be provided. Furthermore, information associated with pressure data obtained from pressure sensing component (200, 300, 300′) may be rendered or otherwise indicated through graph (500) in a time frame the differs from the time frame in which data obtained from accelerometer (310) is rendered or otherwise indicated through graph (500).

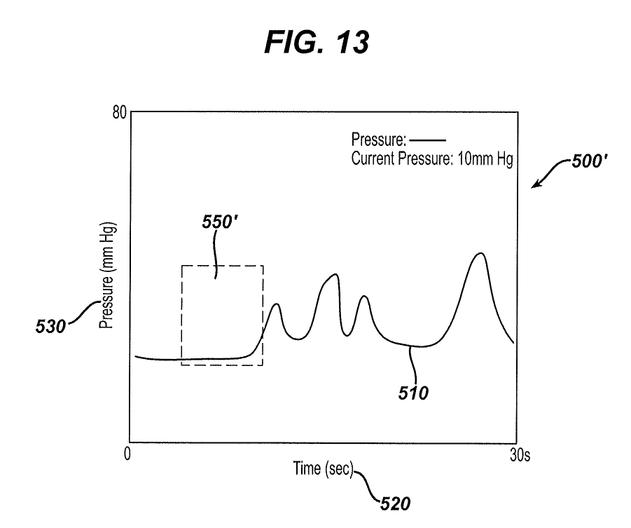

Turning now to FIG. 13, an alternative version of graph (500′) is shown, which may also be part of graphical user interface provided through display device (150). As shown in FIG. 12, portions of graph (500) corresponding to patient movement are highlighted (550) and annotated with, for example, an icon (540). In FIG. 13, portions of graph (500′) corresponding to patient movement are highlighted (550) and “irrelevant” pressure information (e.g., pressure changes caused by the patient changing their orientation by moving from a sitting position to a standing position, etc.) is instead removed or neutralized (e.g., faded, shown in broken lines, shown in a color that differs from the color used to show other pressure data, etc.) to minimize clutter on graph (500′) regarding fluid pressure of gastric band system (10). Such versions of graph (500′) may thus allow the user to focus on other, more pertinent changes in fluid pressure of gastric band system (10) (e.g., changes that are not related to certain movements by the patient). Again, information associated with pressure data obtained from pressure sensing component (200, 300, 300′) and/or data obtained from accelerometer (310) may be rendered or otherwise indicated through graph (500′) in real time, in near-real time, or within any other suitable time frame. Furthermore, information associated with pressure data obtained from pressure sensing component (200, 300, 300′) may be rendered or otherwise indicated through graph (500′) in a time frame the differs from the time frame in which data obtained from accelerometer (310) is rendered or otherwise indicated through graph (500′).

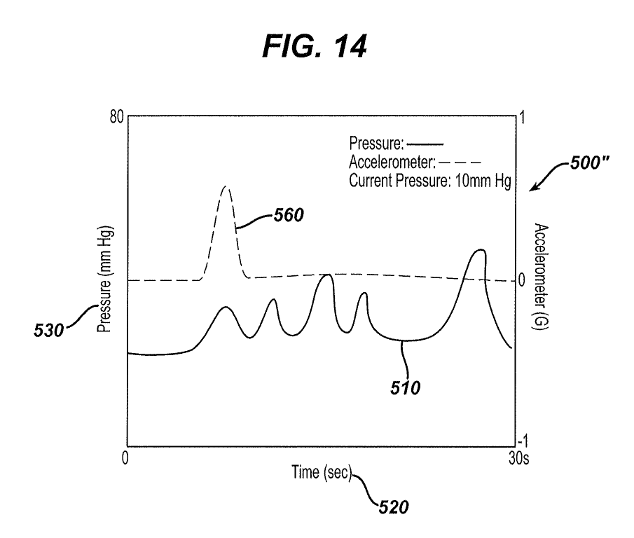

FIG. 14 provides yet another alternative version of graphical user interface where an overlay graph (500″) for accelerometer data is provided to allow simultaneous viewing with pressure data. Time (520) is represented by horizontal axis. Pressure (530) is represented by a solid line (510) and is measured on the left vertical axis. Accelerometer data is represented by a dotted line (560) and is measured on right vertical axis. Lines (510, 560) may be generated substantially simultaneously, and may represent pressure data and accelerometer data that are being sensed substantially simultaneously. Accelerometer data is thus viewable in conjunction with substantially contemporaneous pressure data in such a way that the user may view both measurements together. Again, information associated with pressure data obtained from pressure sensing component (200, 300, 300′) and/or data obtained from accelerometer (310) may be rendered or otherwise indicated through graph (500″) in real time, in near-real time, or within any other suitable time frame. Furthermore, information associated with pressure data obtained from pressure sensing component (200, 300, 300′) may be rendered or otherwise indicated through graph (500″) in a time frame the differs from the time frame in which data obtained from accelerometer (310) is rendered or otherwise indicated through graph (500″).

The foregoing examples have discussed the transmission of pressure data obtained from pressure sensing component (200, 300, 300′) and data obtained from accelerometer (310) to a display device (150). While graphs (500, 500′, 500″) are noted above as ways in which such data may be rendered to a user via display device (150), it should be understood that such data may be rendered to a user in any other suitable fashion, including but not limited to other variations of graphs (500, 500′, 500″) or other forms of user feedback interfaces. It should also be understood that pressure data obtained from pressure sensing component (200, 300, 300′) and data obtained from accelerometer (310) may be transmitted to some other type of processing device, in addition to or in lieu of being transmitted to display device (150). Such an alternative processing device may lack a display (152) altogether. Such an alternative processing device may also be coupled with a display device (150), and may transmit data to a display device (150) after first performing further processing on pressure data obtained from pressure sensing component (200, 300, 300′) and data obtained from accelerometer (310). By way of example only, pressure sensing component (200, 300, 300′) may be coupled with such an alternative processing device via cable (160, 410) (e.g., a USB cable, etc.), wirelessly (e.g., via RF, infrared, etc.), and/or in any other suitable fashion. A display device (150) may extract processed pressure data and/or accelerometer data from the alternative processing device in any suitable fashion, including but not limited to extracting such data via one or more wires/cables and/or wirelessly.

In some versions, an alternative processing device comprises an 8 or 10-bit microcontroller (not shown) that has one or two ADC channels to input an analog output voltage of one or more accelerometers (310) and general purpose I/O pins for displaying the accelerometer data on a computer (e.g., desktop PC, laptop, etc.) through a communication protocol; or on an LCD screen or other type of display. It should also be understood that, in some versions, there is simply no display at all anywhere within the system. For instance, some such versions may just include an I/O channel to send a signal for turning another device on or off when accelerometers (310) are oriented within a particular orientation angle range. Other suitable components and configurations for an alternative processing device will be apparent to those of ordinary skill in the art in view of the teachings herein.

In some versions, regardless of whether graphs (500, 500′, 500″) or variations thereof are also provided through a display device (150), software/firmware on display device (150) and/or software/firmware on some alternative processing device may also use data from accelerometer (310) to compensate for the impact of patient positioning/movement on pressure readings. In other words, display device (150) and/or some alternative processing device may include a logic programmed with an algorithm to process pressure data with compensation using data from accelerometer (310). For instance, such software may use data from accelerometer (310) to compensate for hydrostatic pressure changes when the patient transitions from a lying position (e.g., accelerometer (310) senses a “+1 g” acceleration/orientation) to a sitting or standing position (e.g., accelerometer (310) senses a “0 g” acceleration/orientation). In addition to or in lieu of providing a graphical annotation or other type of visual indication of such changes in patient position through display (152), software/firmware on display device (150) and/or on some alternative processing device may factor hydrostatic pressure changes into a formula to provide a “true” gastric band system (10) pressure reading.

It should be understood that such compensation or “factoring in” of data from accelerometer (310) need not be limited to data indicative of the patient's orientation. By way of example only, data from accelerometer (310) that may be factored into pressure data or otherwise be compensated for in order to provide a “true” gastric band system (10) pressure reading may include swallowing by the patient, heart rate of the patient, breathing rate of the patient, and/or virtually any other physiological parameter of the patient. Accelerometer (310) may thus allow dynamic compensation or filtering of swallowing, heart rate, breathing rate, or virtually any other physiological parameter of the patient that may be picked up by accelerometer (310). The compensation may be displayed to the user (e.g., through a graph (500, 500′, 500″), etc.) or the compensation be simply automatic (e.g., the compensation may be “transparent” to the user, such that only corrected pressure data is shown to the user). Furthermore, such patient physiological parameter data may be acquired from any other suitable source or combination of sources, in addition to or in lieu of being acquired from accelerometer (310). In any event, it should be understood that measurements associated with a patient's orientation, movement, and/or other physiological parameters may be taken and processed independent of a display, particularly when an alternative processing device as described herein is used.

It should also be understood that a display device (150) and/or some other type of processing device that is in communication with pressure sensing component (200, 300, 300′) and/or accelerometer (310) could simply use one or more LEDs and/or other types of visual indication to signal movement, orientation, and/or some other physiological parameter of the patient. In addition or in the alternative, a display device (150) and/or some other type of processing device that is in communication with pressure sensing component (200, 300, 300′) and/or accelerometer (310) could simply use one or more auditory tones and/or other types of auditory indication to signal movement, orientation, and/or some other physiological parameter of the patient.

It will become readily apparent to those skilled in the art that examples described herein may have applicability to other types of implantable bands. For example, bands are used for the treatment of fecal incontinence. One such band is described in U.S. Pat. No. 6,461,292, entitled “Anal Incontinence Treatment with Wireless Energy Supply,” issued Oct. 8, 2002, the disclosure of which is incorporated by reference herein. Bands can also be used to treat urinary incontinence. One such band is described in U.S. Pat. No. 7,621,863, entitled “Urinary Incontinence Treatment with Wireless Energy Supply,” issued Nov. 24, 2009, the disclosure of which is incorporated by reference herein. Bands can also be used to treat heartburn and/or acid reflux. One such band is described in U.S. Pat. No. 6,470,892, entitled “Mechanical Heartburn and Reflux Treatment,” issued Oct. 29, 2002, the disclosure of which is incorporated by reference herein. Bands can also be used to treat impotence. One such band is described in U.S. Pat. No. 7,442,165, entitled “Penile Prosthesis,” issued Oct. 28, 2008, the disclosure of which is incorporated by reference herein. Various ways in which the teachings herein may be incorporated with the teachings of these patent references will be apparent to those of ordinary skill in the art.

It will also be readily apparent to those skilled in the art that examples described herein may have applicability to other types of devices (i.e., not just implantable bands per se). For instance, a syringe (110) and needle (120) fitted with any type of pressure sensing component (200, 300, 300′) described herein may be used to adjust the pressure of fluid within a gastric balloon or other volume occupying device; the pressure of fluid within an infusion port; etc. Various other types of devices and systems with which the examples described herein may be used will be apparent to those of ordinary skill in the art.

Versions of the devices disclosed herein can be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, embodiments of the device may be disassembled, and any number of the particular pieces or parts of the device may be selectively replaced or removed in any combination. Upon cleaning and/or replacement of particular parts, embodiments of the device may be reassembled for subsequent use either at a reconditioning facility, or by a surgical team immediately prior to a surgical procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Versions of the present invention have application in conventional endoscopic and open surgical instrumentation as well as application in robotic-assisted surgery.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. An apparatus comprising:
(a) a body defining a hollow interior portion;
(b) a first port in selective fluid communication with the hollow interior portion of the body;
(c) a second port in fluid communication with the hollow interior portion of the body;
(d) a first sensing device in fluid communication with the hollow interior portion, wherein the first sensing device is configured to sense pressure of fluid within the hollow interior portion; and
(e) a second sensing device coupled with the body, wherein the second sensing device is configured to sense at least one of movement of the body, orientation of the body, or a physical attribute of a patient.

2. The apparatus of claim 1, wherein the first sensing device comprises a pressure sensor located within the hollow interior portion.

3. The apparatus of claim 1, wherein the second sensing device comprises an accelerometer.

4. The apparatus of claim 1, wherein the second sensing device is located in the body.

5. The apparatus of claim 1, further comprising a connector, wherein the connector comprises an interface portion configured to removably connect with the body, wherein the connector is further configured to couple with an external display device.

6. The apparatus of claim 5, wherein the connector comprises a cable coupled with the interface portion, wherein the cable is configured to communicate data from the first sensing device and the second sensing device.

7. The apparatus of claim 6, further comprising an external display device coupled with the cable, wherein the external display device includes a logic configured to simultaneously display data from the first sensing device and data from the second sensing device as a function of time.

8. The apparatus of claim 5, wherein the second sensing device is located within the interface portion of the connector.

9. The apparatus of claim 5, wherein the first sensing device is located within the interface portion of the connector.

10. The apparatus of claim 1, wherein the first port is configured to couple with a syringe, wherein the second port is configured to couple with a needle.

11. A system for measuring pressure within an implantable restriction device, the system comprising:
(a) a pressure sensor, wherein the pressure sensor is configured to measure pressure data associated with the implantable restriction device;
(b) an accelerometer or gravitometer configured to measure at least one of movement of the implantable restriction device, orientation of the implantable restriction device, or a physical attribute of a patient; and
(c) a processing device configured to receive and process data associated with the pressure sensor and the accelerometer or gravitometer, wherein the processing device is further configured indicate movement or orientation of the implantable restriction device corresponding with the pressure data.

12. The system of claim 11, wherein the processing device comprises a display device having a display configured to indicate movement or orientation of the implantable restriction device corresponding with the pressure data by providing graphical representations associated with movement or orientation data from the accelerometer or gravitometer, wherein the representations associated with movement or orientation data comprise graphical representations of a human body, wherein the graphical representations depict positions of a human body associated with orientation of the implantable restriction device.

13. The system of claim 11, wherein the processing device comprises a display device having a display configured to only show pressure data associated with the implantable restriction device for pressure data that corresponds to movement data where the movement data remains within a predetermined threshold.

14. An apparatus, comprising:
(a) an implantable restriction device;
(b) an adjustment device in communication with the implantable restriction device, wherein the adjustment device is configured to adjust restrictiveness of the implantable restriction device;
(c) a first sensor in communication with the implantable restriction device, the first sensor being configured to measure a first parameter associated with the implantable restriction device;
(d) a second sensor in communication with the implantable restriction device, the second sensor being configured to measure a second parameter associated with the implantable restriction device; and
(e) an output device configured to render a plurality of data points corresponding to measurements from the first sensor in relation to effects of the second parameter on the first parameter.

15. The apparatus of claim 14, wherein the first parameter is an internal pressure associated with the implantable restriction device and wherein the first sensor is a pressure sensor.

16. The apparatus of claim 14, wherein the second parameter is movement or orientation associated with the implantable restriction device and wherein the second sensor is selected from the group consisting of: an accelerometer, a plurality of accelerometers, a multi-axis accelerometer, a gravitometer, and combinations thereof.

17. The apparatus of claim 14, wherein the output device comprises a display device having a display configured to render graphical notations to depict the effect of the second parameter on the first parameter.

18. The apparatus of claim 14, wherein the output device comprises a display device having a display configured to simultaneously plot the first parameter and the second parameter as a function of time.

19. The apparatus of claim 14, wherein the implantable restriction device comprises a gastric band.

20. The apparatus of claim 19, wherein the adjustment device comprises a syringe configured to modify a volume of fluid within the gastric band to change the restrictiveness of the gastric band.
